(19) United States
(12) Patent Application Publication
(10) Pub. No.: US 2010/0016765 A1
(43) Pub. Date: Jan. 21, 2010

(54) INTEGRATED SPECT IMAGING AND ULTRASOUND THERAPY SYSTEM

(75) Inventors: Christopher S. Hall, Hopewell Junction, NY (US); Shunmugavelu Sokka, Brighton, NY (US); Balasundara I. Raju, Tarrytown, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/520,432

(22) PCT Filed: Dec. 19, 2007
(86) PCT No.: PCT/IB07/55240

§ 371 Date: Jun. 19, 2009

Related U.S. Application Data
(60) Provisional application No. 60/871,263, filed on Dec. 21, 2006.

(51) Int. Cl. A61N 7/00; A61M 5/00; G01T 1/164; G01T 1/20

(52) U.S. Cl. 601/2; 604/22; 250/363.04; 250/362

(57) ABSTRACT

A method and apparatus for the integration of an ultrasound transducer with a SPECT imaging system is disclosed. The ultrasound transducer allows the delivery of particle-born drug therapies or thermally active therapies at the same site detected with SPECT imaging. The system includes a SPECT imaging subsystem; an ultrasound transducer for producing a signal representative of applied ultrasound and signally coupled to the SPECT imaging subsystem; and co-registering means for co-registering the signal representative of applied ultrasound in a coordinate system of SPECT imaging subsystem. The SPECT subsystem may be replaced with a SPECT/CT combined system to provide morphology that allows for treatment planning. In another embodiment, the ultrasound transducer can be used intermittently with the sequential gathering of SPECT images for tracking time-varying properties of the binding of specific SPECT contrast agents to biomarkers of disease. In this way, the ultrasound and SPECT imaging devices form a novel combination to improve patient care through improved spatial registration, therapy planning, and workflow.

### TECHNICAL FIELD

The present disclosure relates to an apparatus and method for integrating single photon emission computed tomography (SPECT) with an ultrasound transducer.

### BACKGROUND

Molecular imaging techniques are revolutionizing the clinical methods of diagnosis and staging of specific pathologies. In these techniques, an imaging modality-specific contrast agent is generally conjugated with the complement for a biological marker of disease. These markers are usually proteins expressed during the various stages of disease, although they can also sometimes be the altered cell metabolism associated with pathological cells. The contrast agents are specific to each modality. One of the promising imaging modalities for molecular imaging's future is single photon emission computed tomography (SPECT). SPECT also has the capability of modelling the flow of fluids in three dimensions, which renders it capable of imaging both blood flow and the delivery of therapeutic drugs to target locations in organs of the body.

Another modality which is useful for both imaging and therapeutic interventions is ultrasound. More particularly, the use of high intensity focused ultrasound is currently being used as an approach for thermal therapeutic intervention for uterine fibroids and has been examined for possible uses in the treatment of liver, brain, and other cancerous lesions. In addition, ultrasound has also been the subject of much research as a means of mediating clot dissolution (sonothrombolysis), drug delivery, and gene therapy. The use of ultrasound in all of these applications is desirable because it allows non-invasive treatment of deep tissues with little or no effect on overlying organs.

Increased detection techniques can be complemented or combined with novel approaches to therapies. However, coordinating an imaging technique, such as CT scans, which provide accurate images in a well defined coordinate system, with a manual therapeutic delivery system, can present significant difficulties. Because the ultrasound coordinates do not match the CT scan coordinates, and the ultrasound probe is moving relative to the CT scanner, the placement of the therapeutic zone is not always apparent in an ultrasound image or CT scan image.

What would be desirable are apparatus and methods that effectively combine the sensitivity of SPECT imaging with non-invasive approaches to therapy. These and other objects are satisfied by the systems and methods of the present disclosure.

### SUMMARY

The present disclosure relates to a method and apparatus for integrating a single photon emission computed tomography (SPECT) subsystem with an ultrasound transducer. An exemplary embodiment of the present disclosure includes a SPECT imaging subsystem; an ultrasound transducer for producing a signal representative of applied ultrasound and signally coupled to the SPECT imaging subsystem; and co-registering means for co-registering the signal representative of applied ultrasound in a coordinate system of the SPECT imaging subsystem. Exemplary embodiments of the disclosed system also include driving electronics for controlling an ultrasonic beam produced by transducer elements of the ultrasound transducer, one or more amplifiers for amplifying a received signal from the ultrasound transducer, and a gantry/patient bed system for receiving a reclining patient and for co-registering the ultrasound transducer and the SPECT imaging subsystem, whether alone or in combination with other co-registration structures and components.

The disclosed ultrasound transducer is generally adapted to deliver therapeutic ultrasound to specific diseased tissue while tracking the distribution of the therapeutic ultrasound in the coordinate system of the SPECT imaging system. The SPECT subsystem may be replaced with a SPECT/CT combined system to provide morphology that allows for treatment planning. In another embodiment, the ultrasound transducer can be used intermittently with the sequential gathering of SPECT images for tracking time-varying properties of the binding of specific SPECT contrast agents to biomarkers of disease.

Additional features, functions and advantages of the disclosed systems and methods will be apparent from the detailed description which follows, particularly when read in conjunction with the accompanying figures.

### BRIEF DESCRIPTION OF THE DRAWINGS

For a more complete understanding of the present disclosure, reference is made to the following detailed description of exemplary embodiments considered in conjunction with the accompanying drawings, in which:

FIG. 1 is a schematic diagram of an exemplary system which integrates SPECT imaging equipment with an ultrasound transducer in accordance with an embodiment of the present disclosure;

FIG. 2 is a schematic diagram of a set of ultrasound transducer elements which steer a beam of ultrasound toward a focal zone in the body of a patient using the exemplary system of FIG. 1;

FIG. 3 is a perspective view of an ultrasound transducer registered in the coordinate system of the SPECT imaging subsystem of FIG. 1;

FIG. 4 is a schematic diagram depicting how the ultrasound transducer of FIG. 1 can deliver therapeutic ultrasound to specific diseased tissue; and

FIG. 5 is a schematic diagram illustrating how an ultrasound transducer can be used intermittently with the sequential gathering of SPECT images according to an alternative embodiment of the present disclosure.

### DESCRIPTION EXEMPLARY EMBODIMENT(S)

The present disclosure is directed to advantageous integration and co-registration of an ultrasound transducer with a SPECT imaging system. The disclosed SPECT imaging system may be employed with a wide variety of contrast agents to provide highly sensitive and specific detection of disease. The disclosed ultrasound transducer allows the delivery of particle-born or microbubble-based drug therapies, or thermally active therapies at the same site detected with SPECT imaging. The integration of these two modalities may be used to dramatically improve patient care through, inter alia, improved spatial registration, therapy planning, and workflow.

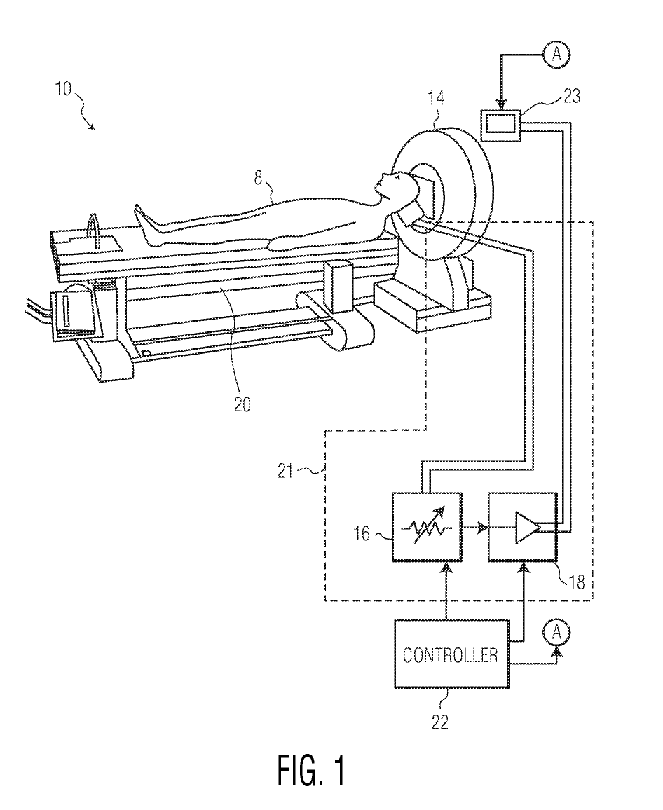

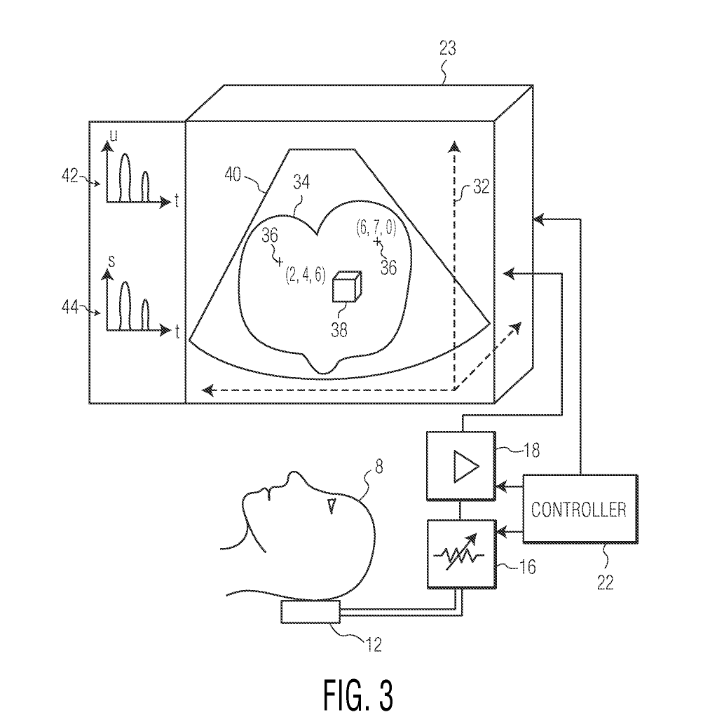

With reference to FIG. 1, a schematic diagram of an exemplary system which integrates a SPECT imaging equipment with an ultrasound transducer according to the present disclosure is shown, generally indicated at 10. The system 10 includes an ultrasound transducer 12, a SPECT imaging subsystem 14, driving electronics 16 for controlling an ultrasonic beam (see FIG. 3) produced by transducer elements of the ultrasound transducer 12, one or more amplifiers 18 for amplifying a received signal from the ultrasound transducer 12, and a gantry/patient bed system 20 for receiving a reclining patient 8 and for co-registering the ultrasound transducer 12 and the SPECT imaging subsystem 14. The ultrasound transducer 12, the driving electronics 16, and the amplifier(s) 18 generally constitute an ultrasound subsystem 19. Additional components may be included in the ultrasound subsystem, as is well known to persons skilled in the art.

Exemplary system 10 can also include a controller 22 which controls and synchronizes operations of the SPECT imaging subsystem 14 and ultrasound subsystem 19. System 10 can also include a user interface 23, e.g., a keyboard/processor/monitor, which provides controls for the clinician to set the parameters related to both the SPECT imaging subsystem 14 and ultrasound subsystem 19. The monitor may also be used to display timing synchronization between the two subsystems. The controller 22 can optionally be provided with functionality, e.g., computer programming, to automatically move ultrasound transducer 12 (which could be placed on optional positioning systems (not shown)) to a desired region of the patient's body based on data received from the SPECT imaging subsystem 14 or other source. Ultrasound transducer 12 can be placed beneath the table 20 on which the patient 8 is lying, although other locations may be employed, as will be readily apparent to persons skilled in the art.

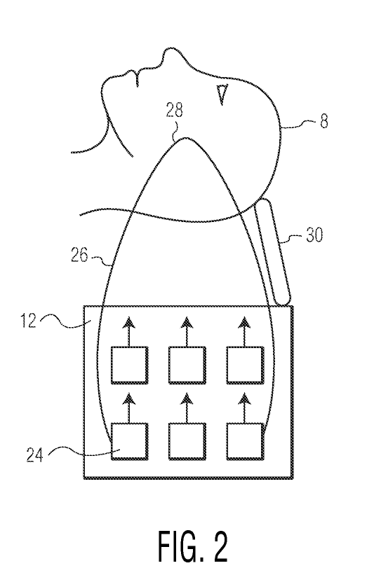

Referring now to FIG. 2, the ultrasound transducer 12 can include multiple ultrasound transducer elements 24, e.g., piezo-electric transducers, which are capable of steering an ultrasonic beam in either two or three dimensions. The transducer elements 24 are generally effective in establishing a focal zone 28 for therapy. The ultrasound transducer 12 is coupled to a portion of the body of a patient 8 via a tissue-coupling medium 30. Referring now to FIG. 3, the ultrasound transducer 12 can be registered in the coordinate system 32 of the SPECT imaging subsystem 14. The display 23 of the SPECT imaging subsystem 14 captures a volumetric image 38 of the pathological tissue. Specific points 36 for two dimensional ultrasound or specific volumes 38 for three dimensional ultrasound can be simultaneously displayed, i.e., the ultrasound transducer beam (not shown) of the ultrasound subsystem 19 can be co-registered with the SPECT imaging coordinate system 32. Optionally, the ultrasound image 40 can be displayed or co-registered with the SPECT imaging coordinate system 32 on the display 23. Timing data 42, 44 for the ultrasound signal and the SPECT image can also be displayed simultaneously on the display 23.

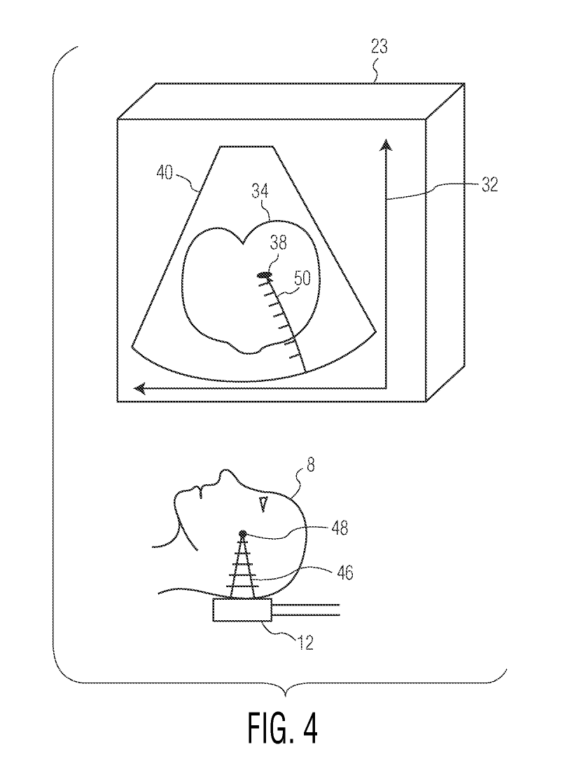

Referring now to FIG. 4, the ultrasound transducer 12 can deliver therapeutic ultrasound 46 to specific diseased tissue 48. For example, the therapeutic ultrasound 46 can be used to deliver drug-bearing contrast agents, thermally activated drugs, or deliver heat energy for thermal sensitization of tissue for concomitant radiotherapy treatments. In this way, ultrasound mediated therapy can alter the pathological tissue 48. The delivery of such therapeutic agents can be viewed in near real time (see path 50) using display 23.

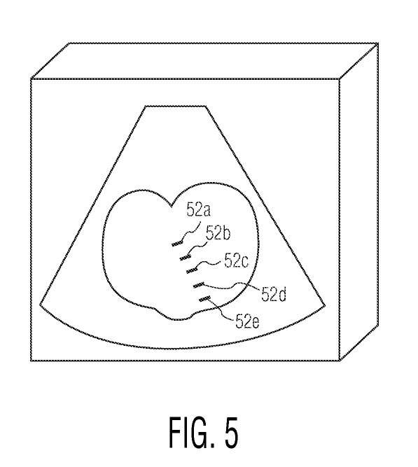

According to another illustrative aspect of the present disclosure, the SPECT subsystem may be replaced with a SPECT/CT combined system. The CT imaging component can provide morphology that allows for treatment planning (acoustic windows, absorbing material in the path, etc.) with ultrasound therapy. Now referring to FIG. 5, an ultrasound transducer can be used intermittently with the sequential gathering of SPECT images 52a-52e, which can then be used to track time-varying properties of the binding of specific SPECT contrast agents to biomarkers of disease. In this way, immediate feedback is provided to the treating physician of the efficacy of the ultrasound mediated therapy.

The present disclosure has numerous advantageous applications. For example, system 10 may be employed for SPECT molecular imaging approaches where an immediate site-specific therapy is desired. SPECT imaging provides a sensitive technique for imaging of cellular function and targeted molecular markers in vivo. The combination of ultrasound therapy and SPECT imaging in a registered and integrated system allows for both immediate treatment of the detected zone and a means of measuring treatment outcome in a rapid manner, as biomarkers of the disease are altered through ultrasound-mediated therapy and monitored through alterations in the kinetics of the SPECT tracers. Having an integrated system—rather than separate SPECT and ultrasound therapy systems—is highly advantageous for a number of reasons, including enabling the registration of SPECT data with the ultrasound therapy, enabling synchronization between imaging and therapy, especially when repeated therapy and imaging operations are done, simplifying the user interface for the clinician, and improving workflow in a clinical environment.

It will be understood that the embodiments described herein are merely exemplary and that a person skilled in the art may make many variations and modifications without departing from the spirit and scope of the invention. All such variations and modifications are intended to be included within the scope of the invention.

1. An apparatus for integrating a single photon emission computed tomography (SPECT) subsystem with an ultrasound transducer, comprising:
a SPECT imaging subsystem;
an ultrasound transducer for producing a pressure field representative of an applied ultrasound signal and coupled to the SPECT imaging subsystem; and
co-registering means for co-registering said signal representative of applied ultrasound in a coordinate system of the SPECT imaging subsystem.

2. The apparatus of claim 1, wherein said co-registering means includes, at least in part, a gantry/patient bed system.

3. The apparatus of claim 2, further comprising an automatic positioning system coupled to the ultrasound transducer.

4. The apparatus of claim 3, further comprising a controller for moving the ultrasound transducer using the automatic positioning system to a desired region of a patient's body based on data received from the SPECT imaging subsystem.

5. The apparatus of claim 1, further comprising a user interface for providing controls for setting parameters related to both said SPECT imaging subsystem and said ultrasound transducer and for displaying the timing synchronization and spatial location integration between the two subsystems.

6. The apparatus of claim 1, further comprising a display that is adapted to display graphical information representative of said ultrasound pressure field in the coordinate system of a SPECT image.

7. The apparatus of claim 1, wherein said ultrasound transducer comprises a plurality of transducer elements capable of steering an ultrasonic beam which can produce one of a two dimensional image and three dimensional image in said coordinate system of said SPECT imaging subsystem.

8. The apparatus of claim 1, further comprising driving electronics for controlling an ultrasonic beam produced by transducer elements of the ultrasound transducer and one or more amplifiers for amplifying a received signal from the ultrasound transducer.

9. The apparatus of claim 1, further comprising a computer tomography (CT) scanner integrated with the SPECT imaging subsystem for providing morphology for allowing for treatment planning with ultrasound therapy.

10. The apparatus of claim 1, further comprising means for allowing said ultrasound transducer to be used intermittently with sequential gathering of SPECT images.

11. The apparatus of claim 1, wherein said ultrasound transducer can deliver therapeutic ultrasound.

12. The apparatus of claim 11, wherein said therapeutic ultrasound delivers one of drug-bearing contrast agents, thermally activated drugs, and heat energy for thermal sensitization of tissue.

13. A method for integrating a single photon emission computed tomography (SPECT) subsystem with an ultrasound transducer, comprising the steps of:
providing a SPECT imaging subsystem;
providing an ultrasound transducer for producing a pressure field representative of an applied ultrasound signal;
signally coupling said ultrasound transducer to the SPECT imaging subsystem; and
co-registering said signal representative of applied ultrasound in a coordinate system of SPECT imaging subsystem.

14. The method of claim 13, further comprising the step of moving the ultrasound transducer to a desired region of a patient's body based on data received from the SPECT imaging subsystem.

15. The method of claim 13, further including the step of providing controls for setting parameters related to both said SPECT imaging subsystem and the ultrasound transducer and displaying the timing synchronization and spatial location between the two subsystems.

16. The method of claim 13, further including the step of steering an ultrasonic beam which can produce one of a two dimensional image and three dimensional image in the coordinate system of said SPECT imaging subsystem.

17. The method of claim 13, further including the step of providing a computer tomography (CT) scanner integrated with the SPECT imaging subsystem for providing morphology for allowing for treatment planning with ultrasound therapy.

18. The method of claim 13, further including the step of allowing the ultrasound transducer to be used intermittently with sequential gathering of SPECT images.

19. The method of claim 13, further including the step of delivering therapeutic ultrasound using the ultrasound transducer.
